(19) United States
(12) Patent Application Publication
(10) Pub. No.: US 2025/0186524 A1
(43) Pub. Date: Jun. 12, 2025

(54) SYNTHESIS OF A CARBON QUANTUM DOTS AND SILVER NANOPARTICLE COMPOSITION

(71) Applicant: KING FAISAL UNIVERSITY, AL HASA (SA)

(72) Inventors: MAI MOSTAFA KHALAF ALI, AL HASA (SA); HANY MOHAMED ABD EL-LATEEF AHMED, AL HASA (SA); MOHAMED GOUDA, AL HASA (SA); MALLAK MEGALEA ZIKRY, GIZA (EG); M.S. AMIN, CAIRO (EG); KHADIJAH M. EMRAN, AL-MOUNAWARA (SA); AHMED M. ABU-DIEF, AL-MOUNAWARA (SA)

(21) Appl. No.: 18/537,735

(22) Filed: Dec. 12, 2023

(51) Int. Cl. A61K 36/185; A61K 9/51; A61K 33/44; A61P 31/04; A61P 31/10; A61P 35/00; A61P 39/06; B82Y 5/00

(52) U.S. Cl. CPC A61K 36/185; A61K 9/5115; A61K 33/44; A61P 31/04; A61P 31/10; A61P 35/00; A61P 39/06; B82Y 5/00

(57) ABSTRACT

A method of preparing a carbon quantum dots (CD) and Moringa oleifera silver nanoparticle (CD-MOE-AgNP) composition includes providing a Moringa oleifera extract, combining the Moringa oleifera extract with silver nitrate to provide silver nanoparticles, and combining the silver nanoparticles with the carbon quantum dots (CD) to provide the CD-MOE-AgNP composition. In an embodiment, combining the carbon quantum dots (CD) with the Moringa oleifera silver nanoparticle (CD-MOE-AgNP) composition can include ultra-sonification of the carbon quantum dots (CD) with the Moringa oleifera silver nanoparticles.

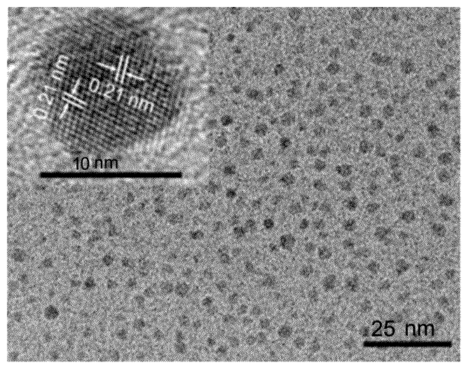

### BACKGROUND

### 1. Field

The disclosure of the present patent application relates to silver nanoparticle compositions, and particularly, to a silver nanoparticle composition synthesized using Moringa oleifera.

### 2. Description of the Related Art

Recently, nanoparticles have demonstrated important uses in a variety of fields. Nanoparticles have been used in electronics, sensing, optics, and medicine, for example.

Synthesis of nanoparticles has been achieved by a variety of methods, including physicochemical, thermal decomposition, electrochemical, microwave assisted, sonochemical, solvothermal, photosynthesis, photochemical reduction, chemical reduction, and continuous-flow methods. These methods are often costly or produce by-products that pose increased risks to human health and the environment.

In recent years, green or environmentally friendly chemical methods have been developed to prepare nanoparticles using plant extracts. Green chemistry has the advantage of being safer, faster, environmentally friendly, and economical. However, the rise of green methods of preparing nanoparticles has also demonstrated that the activities and characteristics of the nanoparticles vary significantly, depending upon the detailed method of synthesis and specific plant extract used.

Moringa oleifera is commonly known as the drumstick tree of the Moringaceae family. It is a native of India and currently grown widely in many tropical and sub-tropical countries. Traditionally, Moringa oleifera was considered to be one of the most therapeutically useful trees, as almost all the parts (pods, leaves, flowers, roots and bark) have been reported to have a gamut of medicinal and nutritional properties. Moringa leaves are a good source of natural antioxidant due to the presence of various types of antioxidant compounds such as ascorbic acid, flavonoids, phenolics and carotenoids. These bioactive compounds are thought to be found in Moringa leaves and have been widely used in various studies, such as wound healing, anti-tumor, anti-fertility, hypotensive, antipyretic, antihepatotoxic, antiepileptic, anti-inflammatory, antiulcer, diuretic hypocholesterolemic, antifungal, antibacterial and anti-cardiovascular and anti-diabetic agents.

Carbon quantum dots (C-dots) are sphere-shaped nanoparticles having high biocompatibility and luminescent properties.

Thus, a silver nanoparticle composition synthesized using an environmentally friendly method solving the aforementioned problems are desired.

### SUMMARY

The present subject matter relates to eco-friendly methods for preparing a silver nanoparticle composition. In one aspect, the present subject matter relates to a method of preparing a carbon quantum dots (CD) and Moringa oleifera silver nanoparticle (CD-MOE-AgNP) composition. The CD-MOE-AgNP composition may be synthesized by providing a Moringa oleifera extract, combining the Moringa oleifera extract with silver nitrate to provide silver nanoparticles, and combining the silver nanoparticles with carbon quantum dots (CD) to provide the CD-MOE-AgNP composition. In an embodiment, combining the carbon quantum dots (CD) with the Moringa oleifera silver nanoparticle (CD-MOE-AgNP) composition can include ultra-sonification of the carbon quantum dots (CD) with the Moringa oleifera silver nanoparticles.

According to an embodiment, the present subject matter relates to a Moringa oleifera silver nanoparticle (CD-MOE-AgNP) composition prepared by the methods as described herein.

According to an embodiment, the present subject matter relates to a pharmaceutical composition comprising the Moringa oleifera silver nanoparticle (CD-MOE-AgNP) composition and a pharmaceutically acceptable carrier.

According to an embodiment, the present subject matter relates to a method of inhibiting microbial growth in a subject, comprising administering to a subject in need thereof a therapeutically effective amount of the pharmaceutical composition as described herein.

According to an embodiment, the present subject matter relates to a method of treating cancer in a subject, comprising administering to a subject in need thereof a therapeutically effective amount of the pharmaceutical composition as described herein.

These and other features of the present subject matter will become readily apparent upon further review of the following specification and drawings.

### BRIEF DESCRIPTION OF THE DRAWINGS

The sole drawing FIGURE is a transmission electron microscopy (TEM) image showing the CD-MOE-AgNP composition with high crystallinity and low particle size (5 nm-8 nm).

### DETAILED DESCRIPTION OF THE PREFERRED EMBODIMENTS

The following definitions are provided for the purpose of understanding the present subject matter and for construing the appended patent claims.

### Definitions

It should be understood that the drawings described above or below are for illustration purposes only. The drawings are not necessarily to scale, with emphasis generally being placed upon illustrating the principles of the present teachings. The drawings are not intended to limit the scope of the present teachings in any way.

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

A “subject” herein is typically a human. In certain embodiments, a subject is a non-human mammal. Exemplary non-human mammals include laboratory, domestic, pet, sport, and stock animals, e.g., mice, cats, dogs, horses, and cows. As used herein, the term “patient” refers to any single subject for which treatment is desired. In certain embodiments, the patient herein is a human. A subject can be considered to be in need of treatment.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

The present subject matter relates to a method of preparing a carbon quantum dot (CD) and Moringa oleifera silver nanoparticle (CD-MOE-AgNP) composition. The CD-MOE-AgNP composition may be synthesized by providing a Moringa oleifera extract, combining the Moringa oleifera extract with silver nitrate to provide silver nanoparticles, and combining the silver nanoparticles with the carbon quantum dots (CD) to provide the CD-MOE-AgNP composition. The CD-MOE-AgNP composition can have high crystallinity and a particle size ranging from about 5 nm to about 8 nm, as shown in the sole drawing FIGURE.

In one embodiment, the extract may be synthesized by harvesting Moringa oleifera leaves, drying the Moringa oleifera leaves, powdering the dried Moringa oleifera leaves, mixing the powdered Moringa oleifera leaves with a solvent to provide a solution, and extracting the solution to provide a Moringa oleifera extract (MOE). The silver nitrate can be added to the extract to provide a mixture including the Moringa oleifera silver nanoparticles. In an embodiment, the powdered Moringa oleifera leaves may be mixed with an appropriate solvent, such as by way of non-limiting example, ethanol.

According to an embodiment, the CD-MOE-AgNP composition can be prepared by mixing the MOE-AgNPs with carbon quantum dots. In an embodiment, the mixing can include ultra-sonication. According to an embodiment, the carbon quantum dots (CD) can be prepared by subjecting lemon juice to hydrothermal treatment to provide the carbon quantum dots. In an embodiment, the hydrothermal treatment can include heating the lemon juice in an autoclave at a temperature ranging from about 100° C. to about 300° C. for about 10 hours to about 15 hours. In an embodiment, the lemon juice can be heated at a temperature ranging from about 120° C. to about 280° C. for about 12 hours.

An embodiment of the present subject matter is directed to a pharmaceutical composition comprising the CD-MOE-AgNP composition and a pharmaceutically acceptable carrier.

An embodiment of the present subject matter is directed to a method of making a pharmaceutical composition including mixing the CD-MOE-AgNP composition with a pharmaceutically acceptable carrier. For example, the method of making a pharmaceutical composition can include mixing the CD-MOE-AgNP composition under sterile conditions with a pharmaceutically acceptable carrier with preservatives, buffers, and/or propellants to create the pharmaceutical composition.

To prepare the pharmaceutical composition, the CD-MOE-AgNP composition, as the active ingredient, is intimately admixed with a pharmaceutically acceptable carrier according to conventional pharmaceutical compounding techniques. Carriers are inert pharmaceutical excipients, including, but not limited to, binders, suspending agents, lubricants, flavorings, sweeteners, preservatives, dyes, and coatings. In preparing compositions in oral dosage form, any of the pharmaceutical carriers known in the art may be employed. For example, for liquid oral preparations, suitable carriers and additives include water, glycols, oils, alcohols, flavoring agents, preservatives, coloring agents, and the like. Further, for solid oral preparations, suitable carriers and additives include starches, sugars, diluents, granulating agents, lubricants, binders, disintegrating agents, and the like.

The present compositions can be in unit dosage forms such as tablets, pills, capsules, powders, granules, ointments, sterile parenteral solutions or suspensions, metered aerosol or liquid sprays, drops, ampules, auto-injector devices or suppositories, for oral parenteral, intranasal, sublingual or rectal administration, or for administration by inhalation or insufflation. The CD-MOE-AgNP composition can be mixed under sterile conditions with a pharmaceutically acceptable carrier and, if required, any needed preservatives, buffers, or propellants. The composition can be presented in a form suitable for daily, weekly, or monthly administration. The pharmaceutical compositions herein will contain, per dosage unit, e.g., tablet, capsule, powder, injection, teaspoonful, suppository and the like, an amount of the active ingredient necessary to deliver an effective dose. A therapeutically effective amount of the CD-MOE-AgNP composition or an amount effective to treat a disease, such as a microbial infection or cancer, may be determined initially from the Examples described herein and adjusted for specific targeted diseases using routine methods.

The CD-MOE-AgNP composition can have antimicrobial, antioxidant, and anti-cancer properties. The CD-MOE-AgNP composition can be administered to a subject in need thereof. In an embodiment, the CD-MOE-AgNP composition can be administered to a subject in need thereof to inhibit bacterial growth. In an embodiment, the CD-MOE-AgNP composition can be administered to a subject to inhibit the growth of E. coli. In an embodiment, the CD-MOE-AgNP composition can be administered to a subject in need thereof to inhibit fungal growth. In an embodiment, the CD-MOE-AgNP composition can be administered to a subject to inhibit the growth of Aspergillus flavus.

An embodiment of the present subject matter is directed to a method of inhibiting microbial growth in a subject, comprising administering to a subject in need thereof a therapeutically effective amount of the pharmaceutical composition according to the present subject matter. In an embodiment, the microbial growth of bacteria. In an embodiment, the bacteria comprises E. coli bacteria. In an embodiment, the microbial growth comprises growth of fungi. In an embodiment the fungi comprises Aspergillus flavus.

An embodiment of the present subject matter is directed to a method of treating cancer in a subject, comprising administering to a subject in need thereof a therapeutically effective amount of the pharmaceutical composition according to the present subject matter. In an embodiment, the cancer is breast cancer.

The CD-MOE-AgNP composition or pharmaceutical compositions thereof can be administered to a subject by any suitable route. For example, the compositions can be administered orally (including bucally and sublingually), nasally, rectally, intracisternally, intra vaginally, intraperitoneally, topically, transdermally (as by powders, ointments, or drops), and/or parenterally. As used herein, “parenteral” administration refers to modes of administration other than through the gastrointestinal tract, which include intravenous, intramuscular, intraperitoneal, intrasternal, intramammary, intraocular, retrobulbar, intrapulmonary, intrathecal, subcutaneous and intraarticular injection and infusion. Surgical implantation may also be contemplated, including, for example, embedding a composition of the disclosure in the body such as, for example, in a tissue, in the abdominal cavity, under the splenic capsule, brain, or in the cornea.

The present teachings are illustrated by the following examples.

### Example 1

### Green Biosynthesized Silver Nanoparticles

A Moringa oleifera extract (MOE) was prepared and 10 mL of the MOE was added to 90 mL of AgNO₃ (2 mM) with constant stirring to provide a mixture. The color of the mixture changed from pale yellow to dark brown within 10 minutes, indicating the formation of silver nanoparticles (MOE-AgNPs), which were then collected by centrifuging.

### Example 2

### Synthesis of Carbon Quantum Dots

40 mL of lemon juice (having an ivory/white color) was put into a Teflon-lined stainless steel autoclave for hydrothermal treatment at temperatures ranging from 120° C. to 280° C. for 12 h. After the reaction, the autoclave was naturally cooled to room temperature. During this hydrothermal process, the ivory/white color of the solution changed to a dark brown color, indicating the formation of the carbon quantum dots. These carbon dots were then purified to remove the larger nanoparticles using 2 μm filter paper.

### Example 3

### Synthesis of the CD-MOE-AgNP Composition

To prepare the CD-MOE-AgNP composition, 300 μl of CD were added to a 100 ml suspension of MOE-AgNPs, ultra-sonicated, stirred, and then filtered to provide the CD-MOE-AgNP composition.

### Example 4

### Anti-Microbial Activity

The CD-MOE-AgNP composition demonstrated potent antibacterial activity against E. coli bacteria with an inhibition zone of 40 mm and a minimum inhibitory concentration (MIC) of 0.75 μg/ml. The standard drug Ofloxacin demonstrated an inhibition zone of 32 mm and an MIC 2.00 μg/ml. The CD-MOE-AgNP composition also demonstrated anti-fungal activity against Aspergillus flavus, with an inhibition zone of 35 mm and an MIC of 1.25 μg/ml. The standard drug Flucazanol demonstrated an inhibition zone of 24 mm and an MIC of 1.50 μg/ml.

To evaluate antimicrobial activity, the bacterial strain (E-coli (-ve)), and fungal strain (Aspergillus flavus), were used. These strains were cultured in nutrient agar and Muller-Hinton medium. Ofloxacin and Fluconazole compounds were used as standard drugs for comparison.

Minimum inhibitory concentrations (MICs) are defined as the lowest concentration of an antimicrobial that will inhibit the visible growth of a microorganism after overnight incubation. MICs are used by diagnostic laboratories mainly to confirm resistance of microorganisms to antimicrobial agents and also to monitor the activity of new antimicrobial agents. MIC was determined in vitro in liquid medium by serial broth dilution method. The MIC values represent the most minuscule concentrations that do not allow for the recognition of any visible growth.

### Example 5

### Anti-Cancer Activity

The CD-MOE-AgNP composition demonstrated super anticancer activity with an IC50 of 1.75 μg/μl against breast cancer cell lines. The Doxorubicin standard drug demonstrated an IC50 of 4.00 μg/μl.

The tests for anti-cancer activity were performed at the National Cancer Institute, Cancer Biology Department, Pharmacology Department, Cairo University. The absorbance or optical density (O.D.) of each well was measured spectrophotometrically at 564 (nm) with an “ELIZA” micro plate reader (Meter tech. Σ960, “USA”). Evaluation of the cytotoxic activity of the prepared nano-complex was carried out against breast cancer cell lines. The evaluation process was carried out in vitro using the Sulfo-Rhodamine-B-stain (SRB). Cells were placed in 96-multiwell plate (10⁴ cells/well) for 24 hrs before processing with the complexes to allow attachment of the cells to the wall of the plate. Various concentrations of the compounds under test in DMSO (0, 1, 2.5, 5 and 10 μM) were added to the cell monolayer. Monolayer cells were incubated with the complexes for 48 hrs at 37° C. and in an atmosphere of 5% CO₂. After 48 hrs, cells were fixed, rinsed, and stained with Sulfo-Rhodamine-B-stain. Excess stain was washed with acetic acid and the attached stain was treated with Tris EDTA buffer. Color intensity was measured in an ELISA reader. IC50 was evaluated and potency was calculated with regard to percentage of change (vistabline standard). The relation between surviving fraction and compound concentration is plotted to get the survival curve of each tumor cell line after the specified compound. The experiment was carried out once and each concentration repeated 3 times.

The inhibitory concentration percent (IC %) was estimated according to the equation: inhibition concentration

$$( IC ) \% = ( Control O . D . - Ligand O . D . ) \times 100 / Control O . D$$

### Example 6

### Anti-Oxidant Activity

The CD-MOE-AgNP composition demonstrated super anti-oxidant activity with IC50 7.5 μg/μl, compared with the l-ascorbic acid standard antioxidant (IC50=50.8 μg/μl).

In vitro antioxidant activity of the composition was evaluated by scavenging the stable DPPH radical modified method. The model of scavenging the stable DPPH radical is a method that is widely used to evaluate antioxidant activities in a relatively short time compared with other methods. DPPH′ radical scavenging test relies on the absorbance change of the radical when deactivated by antioxidants, which are easily observable with the naked eye, as color changes from purple to yellow. Stock solutions of the investigated compounds were dissolved in methanol-DMSO (4:1) which was diluted to final concentrations of 10, 25, 50, 100 and 150. M. Methanolic DPPH (2,2-diphenyl-1-picrylhydrazyl) solution (1 mL, 0.3 mmol) was added to 3.0 mL of the synthesized compounds as well as the standard compound (Ascorbic acid). The tube was protected from light by covering with aluminum foil and the absorbance was measured at 517 nm after 30 min. using methanol as a blank. All the tests were made in triplicates. Vitamin C was used as standard or positive control, parallel to the test compound and in the absence of the test compound/standard used as the negative control. The reduction in the absorbance of DPPH was calculated relative to the measured absorbance of the control. Lower absorbance values of reaction mixture indicated higher free-radical-scavenging activity. The percentage of DPPH radical scavenging activity was calculated using the equation:

$$\% DPPH scavenging activity = \frac{{A}_{C} - {A}_{S}}{{A}_{C}}$$

where A_{C} is the absorbance of the L-ascorbic acid (Standard) and A_{S} is the absorbance of different compounds. The methanolic DPPH solution (1 mL, 0.3 mM) was used as control. The effective concentration of sample required to scavenge DPPH radical by 50% (IC50 value) was obtained by linear regression analysis of dose-response curve plotting between % inhibition and concentrations.

It is to be understood that the CD-MOE-AgNP composition is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A method of synthesizing a carbon quantum dots (CD) and Moringa oleifera silver nanoparticle (CD-MOE-AgNP) composition, comprising:
providing a Moringa oleifera extract and combining the Moringa oleifera extract with silver nitrate to provide Moringa oleifera silver nanoparticles;
preparing carbon quantum dots (CD) by subjecting lemon juice to hydrothermal treatment to provide the carbon quantum dots; and
mixing the carbon quantum dots with the Moringa oleifera silver nanoparticles to provide the CD-MOE-AgNP composition.

2. The method of claim 1, wherein the hydrothermal treatment comprises heating the lemon juice in an autoclave at a temperature ranging from about 100° C. to about 300° C. for about 10 hours to about 15 hours.

3. The method of claim 1, wherein the hydrothermal treatment comprises heating the lemon juice at a temperature ranging from about 120° C. to about 280° C. for about 12 hours.

4. The method of claim 3, wherein mixing the carbon quantum dots with the Moringa oleifera silver nanoparticles comprises ultra-sonication of the carbon quantum dots with the Moringa oleifera silver nanoparticles.

5. A silver nanoparticle composition, prepared by the method of claim 1.

6. The silver nanoparticle composition of claim 5 having a particle size ranging from about 5 nm to about 8 nm.

7. A pharmaceutical composition, comprising the silver nanoparticle composition of claim 5 and a pharmaceutically acceptable carrier.

8. A method of inhibiting microbial growth in a subject, comprising administering to a subject in need thereof a therapeutically effective amount of the pharmaceutical composition claim 7.

9. The method of claim 8, wherein the microbial growth comprises growth of fungi.

10. The method of claim 9, wherein the fungi comprise Aspergillus flavus.

11. The method of claim 8, wherein the microbial growth comprises growth of bacteria.

12. The method of claim 11, wherein the bacteria comprise E. coli.

13. A method of treating cancer in a subject, comprising administering to a subject in need thereof a therapeutically effective amount of the pharmaceutical composition claim 7.

14. The method of claim 13, wherein the cancer is breast cancer.

15. A method of promoting an antioxidant effect in a subject, comprising administering to a subject in need thereof a therapeutically effective amount of the pharmaceutical composition claim 7.
